(19) United States
(12) Patent Application Publication
(10) Pub. No.: US 2026/0048183 A1
(43) Pub. Date: Feb. 19, 2026

(54) MANUAL BREAST PUMP KIT

(71) Applicant: FRIDABABY, LLC, Miami, FL (US)

(72) Inventors: Chelsea HIRSCHHORN, Miami Beach, FL (US); Alaina DEKKER, San Francisco, CA (US); Amanda FLORES, Coral Gables, FL (US); John C. FRITZ, Buffalo, NY (US); Monica TOURNOUX, Miami, FL (US)

(73) Assignee: FRIDABABY, LLC, Miami, FL (US)

(21) Appl. No.: 18/804,486

(22) Filed: Aug. 14, 2024

(51) Int. Cl. A61M 1/06

(52) U.S. Cl. CPC A61M 1/064; A61M 2205/0216; A61M 2205/025; A61M 2210/1007

(57) ABSTRACT

A milk collection vessel with a flexible sidewall and a rigid collar may be attached either to a lever-operated breast pump assembly or to a passive breast pump assembly. The lever-operated assembly has a vessel tube axis offset from the vessel axis to make the lever easier to operate with a small hand. The breast flange of the passive breast pump assembly is formed of an elastomer and, prior to application to the breast, may be everted with the aid of manual tabs.

### BACKGROUND OF THE INVENTION

Manual breast pumps include lever-operated breast pumps and passive breast pumps. In a conventional lever-operated breast pump, the user squeezes a long handle towards a breast pump bottle. The handle has a free end which is grasped by the user, a diaphragm end and a pivot point located in between the free end and the diaphragm end. The diaphragm end is connected, as by a link, to an elastomeric diaphragm that is housed in a diaphragm chamber of a breast pump assembly. The diaphragm bounds a volume that is in fluid communication with an interior space of the breast pump assembly. A typically conical breast flange is applied to the user's breast. A central hole of the breast flange is in fluid communication with the interior space of the breast pump assembly.

The lever-operated breast pump assembly further typically includes a collar for attachment to a milk collection bottle. A duckbill valve selectively communicates the interior space of the assembly to an interior of the collection bottle. When the user squeezes the handle, the diaphragm is lifted, expanding an adjacent volume, and subjecting the interior space and the user's breast to a partial vacuum. This induces the expression of milk. The duckbill valve seals off this partial vacuum from the collection bottle. But, on the reverse stroke of the handle, the duckbill valve opens, permitting the milk to flow into the bottle. For a lever-operated breast pump to work, the handle has to be sufficiently rigid for it to be displaced, around a fulcrum of the assembly, and toward the bottle, and the bottle has to be sufficiently rigid to lever the handle closer to it without it being substantially deformed.

Passive breast pumps, on the other hand, may be molded entirely of elastomeric components. In a typical passive breast pump, an elastomeric breast flange may be everted prior to application to a user's breast, and only thereafter reinverted, to increase a partial vacuum experienced by the user. An elastomeric vessel is squeezed by the user, and is released only after application of the breast flange to the breast, so that a partial vacuum occurs at the breast and encourages milk expression.

It would be advantageous to provide a kit in which a milk collection vessel could be used with both a lever-operated breast pump and a passive breast pump. It would also be advantageous to provide a means for the user to more easily evert a flexible breast flange prior to application to the breast.

### SUMMARY OF THE INVENTION

According to one aspect of the invention, a breast flange has a body formed of an elastomer. A body wall extends from a proximal margin of the body to a distal end thereof. Means are provided for communicating the distal end to a milk storage vessel. At least one tab extends from the outer surface of the body wall to a free end. The body of the breast flange is capable of assuming normal and everted states, both of which are stable. The body wall has a proximal portion and a distal portion. The proximal portion of the body wall, in a normal position, proximally extends from the distal portion to the proximal margin. But in the everted state, the proximal portion assumes an everted position which is outwardly and distally displaced from the normal position, so as to double back on the distal portion of the body wall. A predetermined amount of distal tensile force applied by a user through the tab will evert the body from the normal state to the everted state.

The flange may have two such tabs, each formed on the outer surface of the body wall to be angularly spaced from each other. The user may then use the thumbs and fingers of both hands to evert or fold back the body prior to applying it to her breast.

In one embodiment, the means for communicating the distal end of the flange to the milk storage vessel is a collar whose bottom end is attached to the vessel, and whose top end is attached to the distal end of the milk storage vessel.

According to another aspect of the invention, a manual breast pump kit includes both a lever-operated breast pump assembly and a passive breast pump assembly. The lever-operated breast pump assembly includes a body, a diaphragm and a handle. The body is formed of a first polymer and has a vessel collar. A diaphragm chamber of the body is bounded by the diaphragm, which is formed of a first elastomer. The diaphragm is movable between a first position at which the diaphragm chamber has a first volume, and a second position at which the diaphragm chamber has a second volume larger than the first volume. The body has a fulcrum. The handle is formed of a second polymer and has a free end and a diaphragm end. A pivot of the handle is located between the free and diaphragm ends of the handle and is rotatably coupled to the body fulcrum. A diaphragm link couples the diaphragm end of the handle to the diaphragm and is operable to displace the diaphragm from the first position to the second position. The first and second polymers, as molded, are sufficiently rigid that they will permit a hand of a user to displace the free end of the handle toward the vessel collar to thereby urge the diaphragm from the first position to the second position. The kit further includes a vessel that is selectively attachable to the vessel collar of the lever-operated breast pump assembly. A sidewall of the vessel is formed of a second elastomer that is more flexible than the first or second polymers. The passive breast pump assembly has a flexible breast flange molded of a third elastomer. A proximal end of the flexible breast flange is adapted to fit to a breast of a user. The flexible breast flange has a distal end. A second collar of the passive breast pump assembly is formed from a third polymer that is more rigid than the second elastomer and the third elastomer. The second collar is joined to the flexible breast flange and is operable to be fastened to the vessel so as to communicate the distal end of the flexible breast flange to the vessel interior. When the passive breast pump assembly is fastened to the vessel, the user may squeeze the sidewall of the vessel to create a partial vacuum at the proximal end of the flexible breast flange.

In embodiments, the first elastomer, the second elastomer and/or the third elastomer may be silicone rubber. In embodiments, the silicone rubber compounds used for the first, second and third compounds may have different durometers. The first and/or third polymers may be polypropylene. The second polymer may be ABS or polypropylene.

In one embodiment, the vessel comprises an attachment portion for selective attachment to the vessel collar of the lever-operated breast pump assembly, or to the second collar of the passive breast pump assembly. The vessel further comprises a flexible portion that includes the aforementioned sidewall. The attachment portion is formed of a fourth polymer (such as PPSU) that is more rigid than the second elastomer.

According to a further aspect of the invention, a manual breast pump kit includes a lever-operated breast pump assembly. A body of the breast pump assembly has a breast flange orifice or tube. The kit further includes a plurality of breast pump flanges. Each of the breast pump flanges has a proximal margin for fitting to the user's breast, a flange portion extending distally from the proximal margin to a distal end of the flange portion, and a tube portion distally extending from the distal end of the flange portion to a tube portion end. The tube portion is adapted to be inserted into the breast flange tube of the body. The flange tube portion is connected to the distal end of the flange portion at a transition, whereat a distal end internal diameter is taken. The distal end internal diameter of a first of the plurality of breast flanges is different from the distal end internal diameter of a second of the plurality of breast flanges, such that differences in nipple size are accommodated by different ones of the plurality of breast flanges. The kit may further include a third breast flange and even further breast flanges, all with different distal end internal diameters. In some embodiments, at least some of the provided breast flanges may have a proximal margin that is oval.

According to a still further aspect of the invention, a vessel for containing breast milk comprises an attachment portion molded of a first polymer and a flexible portion molded of an elastomer that is less rigid than the first polymer. The flexible portion is overmolded onto the attachment portion. The flexible portion includes a flexible sidewall which will inwardly elastically deform when squeezed by a hand of a user. The flexible sidewall defines a vessel interior and has an original state. When the vessel interior is in communication with the atmosphere, and when the hand of the user is no longer squeezing the flexible sidewall, the flexible sidewall will return to its original state.

In embodiments, the flexible sidewall may be formed around an axis and may have a top end and a bottom end. A vessel diameter is defined across the inner surface of the flexible sidewall across the axis and at a point about midway between the top and bottom ends of the vessel. The flexible sidewall is capable of being compressed by 95% of adult female users (alternatively: 80% of adult female users) radially inwardly toward the axis by at least 50% of the vessel diameter. In embodiments, the elastomer may be silicone rubber, and/or the first polymer may be polyphenylsulfone.

According to another aspect of the invention, a lever-operated breast pump is provided that has a vessel and a lever-operated breast pump assembly. The assembly has a body, a breast flange and a pumping lever. The body includes a breast flange tube in fluid communication with the breast flange and a vessel tube in fluid communication with the breast flange tube. The body further includes a downwardly hollow vessel collar that is joined to the vessel tube and which is adapted to be affixed to the vessel. An axis of the vessel tube is parallel to but horizontally offset from a vessel axis in a first direction. An outer surface of the vessel tube faces a second direction opposed to the first direction and forms a thumb engagement surface. A lower outer surface of the breast flange tube, an upper surface of the collar and the thumb engagement surface define a thumb space capable of receiving a thumb of the user.

The pumping lever has a free end, a diaphragm end and a fulcrum disposed between the free end and the diaphragm end. A grasping surface of the pumping lever has a first end proximate to the fulcrum and extends from the first end to the free end. The grasping surface is adapted to receive one or more fingers of the user, such that clamping pressure exerted by those one or more fingers will cause the displacement of the grasping surface in the second direction toward the thumb space. Because of the offset of the vessel tube axis from the vessel axis, the distance between a vertical segment of the grasping surface in its resting state to the thumb engagement surface is reduced from what it would be if the vessel tube were coaxial with the vessel axis. This makes the breast pump more ergonomically friendly to users with small hands.

The invention further provides a method for using a passive manual breast pump. An elastomeric breast pump flange is provided to have at least one tab outwardly and distally extending from a location on the outer surface of the breast flange wall. The user applies a predetermined amount of tensile force, in a distal direction, to the at least one tab. Responsive to the step of applying the tensile force, a proximal portion of the breast flange wall is everted, such that the proximal portion is outwardly and distally displaced form a normal position of the proximal portion. The breast flange then is in an everted state. Next, the user squeezes an elastomeric sidewall of a vessel in fluid communication with the breast flange. While squeezing the sidewall, the user applies a locus on an internal surface of the breast flange to the breast, the locus being a proximal end of the breast flange when the breast flange is in the everted state. After the locus is applied, the user exerts force in a proximal direction to the proximal portion of the breast flange wall, reinverting the breast flange to a normal state and sealing the internal surface of breast flange to the breast to thereby maintain a partial vacuum at the breast.

### BRIEF DESCRIPTION OF THE DRAWINGS

Further aspects of the invention and their advantages can be discerned in the following detailed description as read in conjunction with the drawings of exemplary embodiments, in which like characters denote like parts and in which:

FIG. 1 is a perspective view of a manual breast pumping kit according to the invention;

FIG. 2 is a sectional view of a lever-operated breast pump assembly and attached breast pump flange, which are components of the kit shown in FIG. 1;

FIG. 2A is a sectional view of the lever-operated breast pump assembly as attached to a milk collection vessel, showing certain ergonomic relationships;

FIG. 3 is a set of views from a proximal direction of three alternative breast flanges for use with the lever-operated breast pump assembly;

FIG. 4 is an axial sectional view of a vessel for use with the manual breast pumping kit;

FIG. 5 is a bottom perspective view of a passive breast pump assembly for use with the manual breast pumping kit;

FIG. 6A is a sectional view, along a vessel axis, of the passive breast pump assembly shown in FIG. 5, in either a normal or reinverted state;

FIG. 6B is a view along the same sectional plane used for FIG. 6A, showing the flexible breast flange in an everted state;

FIG. 7A is a perspective view of a first step in applying the flexible breast flange to a breast, showing eversion of the flange using tabs;

FIG. 7B is a perspective view of a step occurring after that shown in FIG. 7A, showing the flexible breast flange in an everted stable state;

FIG. 7C shows a step after the step shown in FIG. 7B, showing the application of the everted breast flange to a breast; and

FIG. 7D shows a step after the step shown in FIG. 7C, showing the reinversion of the breast flange to better effect a partial vacuum at the breast.

### DETAILED DESCRIPTION

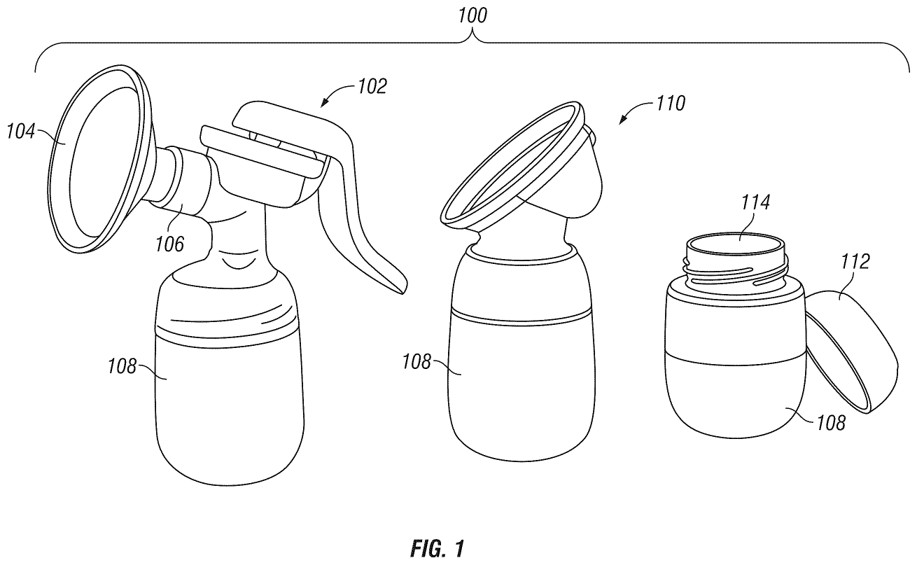

A manual breast pump kit indicated generally by 100 in FIG. 1 includes a lever-operated breast pump assembly 102, at least one breast flange 104 for affixation to a body 106 of the assembly 102, and a collection vessel 108. Three such vessels 108 are shown in FIG. 1 but minimally the kit 100 can have only one such vessel 108. The kit further includes a passive breast pump assembly 110. Either the lever-operated breast pump assembly 102 (alternatively known as an active top), or the passive breast pump assembly 110 (alternatively known as a passive top) may be affixed to the vessel 108 prior to use. In order to store expressed milk, a lid 112 may be used to seal an upper opening 114 of vessel 108.

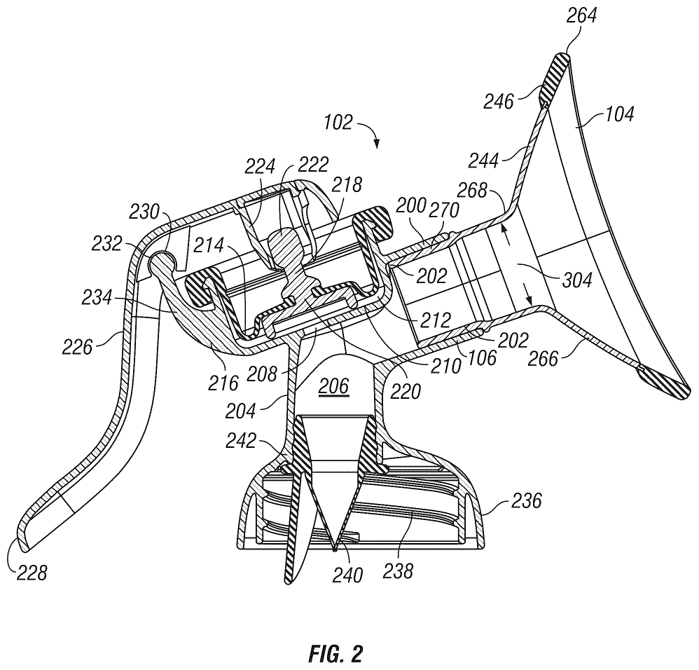

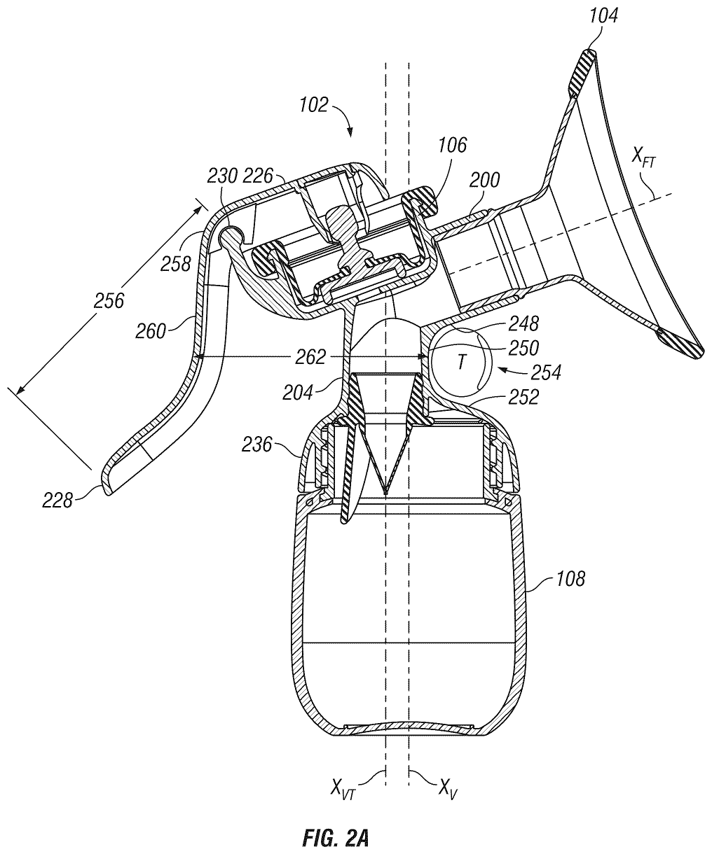

A representative lever-operated breast pump assembly 102 is shown in sectional view in FIG. 2. The assembly body 106 has a breast flange tube 200 which accepts a distal end 202 of a tube portion 203 of the breast flange 104. As used herein, “distal” means farther away from the user's breast, and “proximal” means closer to it. A vessel tube 204 of the body is in fluid communication with the breast flange tube 200. Together, and the illustrated embodiment, inner surfaces of the tubes 200 and 204 define an internal space 206 of the body 106. The tubes 200 and 204 in the illustrated embodiment are cylindrical, but they could take other shapes.

An opening 208 in a wall 210 of body 206 communicates the internal space 206 with a diaphragm chamber 212. A ceiling of the diaphragm chamber is defined by an elastomeric diaphragm 214, which seals to a rigid diaphragm housing 216 to define the chamber 212. FIG. 2 shows the diaphragm 214 in a rest condition, at which a volume of the diaphragm chamber 212 is smallest.

A link 218 has a lower end 220 coupled to diaphragm 214 and an upper end 222 coupled to a diaphragm end 224 of a lever or handle 226. The lever 226 is elongate, has a free end 228 and, disposed between diaphragm end 224 and free end 228, a pivot 230. The pivot 230 may take the form of a horizontally disposed cylinder. The pivot 230 rotates around a fulcrum 232, which may take the form of a horizontally disposed bar with a cylindrical outer surface. The fulcrum 232 may be formed as an upper end of a fulcrum arm 234, itself an extension of diaphragm housing 216.

Vessel tube 204 extends downwardly from tube 200 to a downwardly hollow vessel collar 236, which is adapted to be affixed to the vessel 108. For example, the method of affixing collar 236 to vessel 108 may be female helical threads 238. In the illustrated embodiment, a valve 240, which may be a duckbill valve, is sealably inserted into a lower end 242 of the vessel tube 204.

In operation, the user grasps handle 226 and (in this FIGURE) squeezes handle 226 toward vessel tube 204. This counterclockwise (in this FIGURE) rotation around fulcrum 232 causes the diaphragm end 224 of the lever to lift. Responsive to this, the link 218 moves upward, pulling diaphragm 214 with it. This expands the volume of diaphragm chamber 212. Because of hole 208, this causes a partial vacuum in the internal space 206 and the interior of flange 104, where flange 104 has been sealed to a breast. This induces expression of milk from the breast. However, the partial vacuum is not communicated to the collection vessel 108 (see FIG. 2A), because the valve 240 will stay closed in this condition.

Upon release of handle 226, diaphragm lever end 224 drops and diaphragm 214 reverts to its rest condition, causing the pressure inside internal space 206 to rise. This induces milk collected in internal space 206 to pass through valve 240 and into the interior of vessel 108.

In other embodiments, the pumping action effected by displacing handle 226 may be by means other than a flexible diaphragm. The pump may, for example, be motorized. All aspects of the invention disclosed herein nonetheless apply to such other lever-operated breast pumps.

Diaphragm housing 216, fulcrum arm 234, fulcrum 232, vessel tube 200 and flange tube 200 may be molded as a single unit from a relatively rigid thermoplastic polymer compound, such as one containing polypropylene. Lever 226 may be molded as a single unit from this or another relatively rigid thermoplastic polymer compound, such as one containing acrylonitrile butadiene styrene (ABS) or polypropylene. The valve 240 may be integrally molded of an elastomer such as silicone rubber. Diaphragm 214 may be molded of silicone rubber or of another elastomer. The elastomeric compounds used to mold diaphragm 214 and valve 240 may be the same or may be different from each other in their durometers or other physical properties.

Breast flange 104 may have a substrate 244 formed of a relatively rigid polymer such as polypropylene, and a flange ring 246 overmolded onto substrate 244 out of an elastomer such as a thermoplastic elastomer (TPE).

Breast flange 104 has a proximal margin 264 which in use fits to the user's breast. A flange portion 266 of the breast flange distally extends from margin 264 to a flange portion distal end 268, at which there is a transition to a tube portion 270, which can be substantially cylindrical. The tube portion 270 in turn distally extends from flange portion distal end or transition 268 to the tube portion end 202. Tube portion 270 is adapted to snugly fit within flange tube 200. The flange portion 266 may be conical or, as shown, concavely curved in axial section (i.e, in a plane including axis X_{FT} as seen in FIG. 2A). As viewed end on, the proximal margin 264 may be circular or may be oval. A flange portion distal end diameter 304 is taken at the transition or flange portion distal end 268.

Certain advantageous ergonomic relationships of the lever-operated assembly 102 are illustrated in FIG. 2A. The vessel 108 is formed around a vessel axis X_{V}. A vessel tube axis X_{VT} preferably is not coaxial with axis X_{V} (as is the case of conventional breast pumps of this type), but rather is parallel to and offset from it in a first (here, left) horizontal direction. The spacing between axes X_{V} and X_{VT} may be chosen from the range of about 3 to about 16 mm, and in the illustrated embodiment is about 7 mm.

The flange tube 200 is disposed on an axis X_{FT}, which is at an angle to axis X_{V} such as 105 degrees. The flange tube 200 has a lower outer surface 248 that joins an outer surface 250 of vessel tube 204. Surface 250 acts as, or includes, a thumb engagement surface. In this embodiment, surface 250 is vertical and extends from surface 248 to a top surface 252 of collar 236. Surface 250 faces in a second direction (here, right) that is opposite the first direction of displacement of axis X_{VT} from axis X_{V}. Surfaces 248, 250 and 252 define a thumb space which can comfortably receive a user's thumb T. In one embodiment, surfaces 248 and 252 are sufficiently far apart from each other at their closest point that a thumb T having an approximate diameter of 24 mm may be received in thumb space 254.

A grasping surface 256 of handle 226 is on the outside of handle 226 (that is, facing in the first direction) and extends from a point 258 proximate to pivot 230 to free end 228. At least a segment 260 of grasping surface 256 is vertical or substantially parallel to axis X_{V}. A distance 262 between segment 260 and thumb surface 250 may be in the range of about 60 to about 100 mm, and in the illustrated embodiment is about 70 mm. Because of the offset of the axis X_{VT} of vessel tube 204 from the vessel axis X_{V}, distance 262 may be made small enough to better fit a small hand.

In the illustrated embodiment, breast flange 104 is provided as a piece separate from the lever-operated breast assembly 102. This permits it to be replaced with breast flanges having other designs and/or shapes or dimensions. FIG. 3 illustrates one such possibility. A first breast flange 300 may have an oval shape at its proximal (breast-mating) end and may have an internal diameter 302 across the distal end of its flange portion, at or immediately after the flange body transitions from the flange portion to the tube portion, of about 21 mm. This transition corresponds to transition 268 seen in FIG. 2. Breast flange 104 may have a similar shape at its proximal end 264 but may have an internal diameter 304, corresponding to diameter 302, and measured at transition 268, of about 24 mm. A third breast flange 306 may have an oval shape at its proximal end but may have a corresponding internal diameter 308 at the transition between its tube portion and its flange portion of about 27 mm. Alternatively or in addition, one or more of the flange portions of flanges 104, 300 and 306 may have shapes, proximal to their respective tube portions, which are not oval, such as conically circular shapes, or which are concavely curved rather than conical.

A kit according to the invention may have further breast flanges with other, different flange portion distal end diameters. The nipples of women's breasts come in various sizes and some are larger than others. A woman with relatively large nipples might find breast flange 306 most comfortable to use, while a woman with relatively small nipples might find breast flange 302 most comfortable to use.

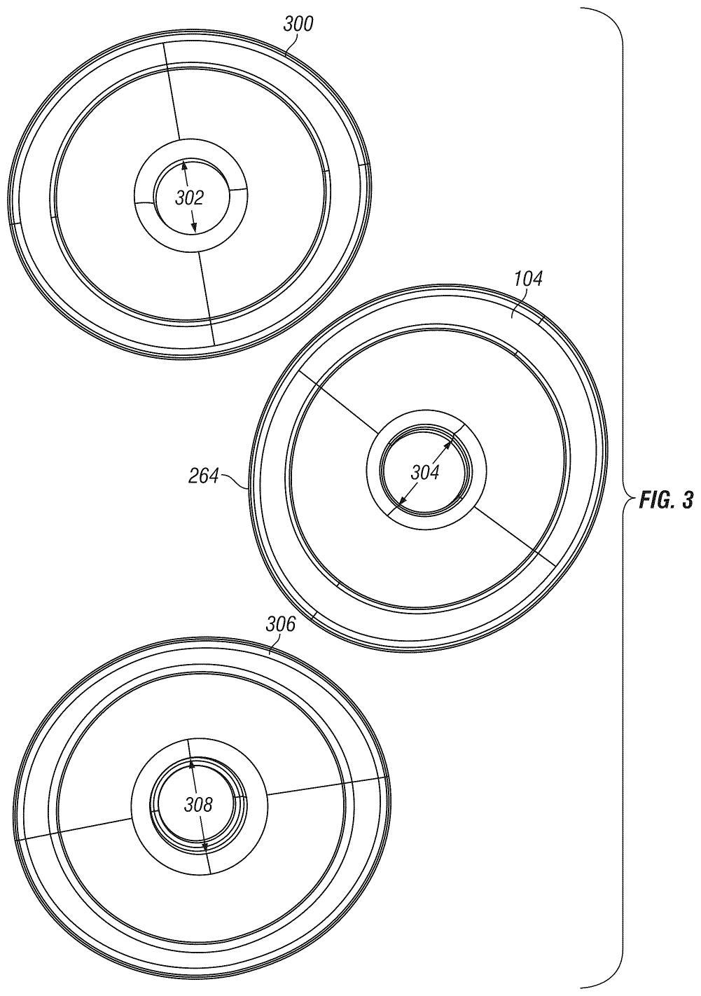

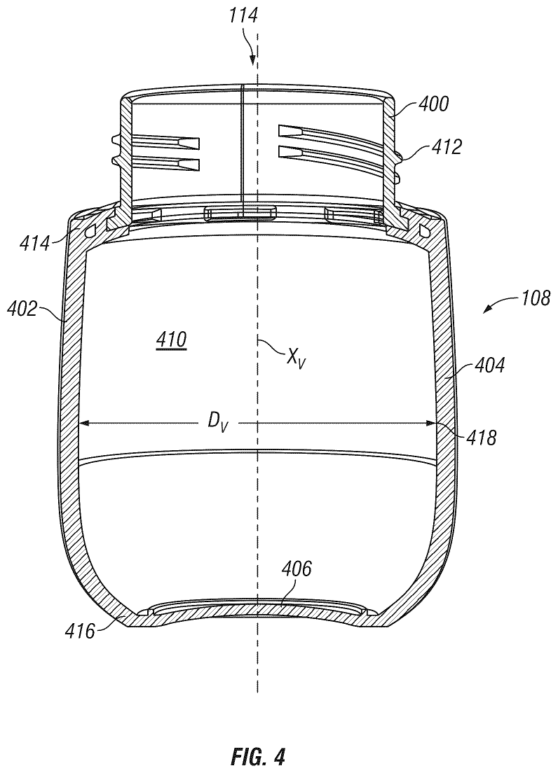

As seen in FIG. 4, the vessel 108 may have a relatively rigid attachment portion 400 for attachment to either the lever-operated breast pump assembly 102 or to the passive breast pump assembly 110. A flexible body portion 402 is attached as by overmolding to the attachment portion 400. The flexible body portion 402 is molded of an elastomer such as silicone rubber, the properties of which may be different from the elastomeric compounds used to mold other elastomeric components described herein. As seen in FIG. 4, the flexible body portion 402 is in an original state, and, once any deflecting or deforming force is removed, body portion 402 will return to the state shown.

The flexible body portion 402 may have a vertically disposed sidewall 404. Sidewall 404 may be about 3 mm thick. Sidewall 404 has a top 414, which is attached to attachment portion 400, and a bottom 416. Sidewall has an internal diameter D_{V} taken at a point 418 about midway between top 414 and bottom 416. The sidewall 404 collapses inwardly toward vessel axis X_{V} upon the application of a predetermined amount of compressive force, as by squeezing with one hand, and then resiliently springs back (or tries to if interior 410 is not in communication with the atmosphere) when this compressive force is removed. Once wall 404 is squeezed and the passive top 110 is applied to a breast, the tendency of the wall 404 to revert to the original state shown here creates a partial vacuum at the breast. But when the interior 410 is communicated to the atmosphere, and once any deflecting force is removed, the wall 404 will revert to its original state. The elastic material used to form wall 404 should have a good shape memory and should not permanently or plastically deform.

The resiliency of wall 404 can be selected such that 95% of adult female users (alternatively: 80% of adult female users), using one hand, will be able to inwardly deform wall 404 toward axis X_{V} by an amount that is at least 50% of vessel diameter D_{V}. The resilience or elasticity of wall 404 should be selected so that its tendency to revert to its original state will create a strong partial vacuum at the breast, but should not be so stiff that a typical user will not be able to substantially decrease the volume of interior 410 by squeezing wall 404 with one hand.

In the illustrated embodiment, the flexible body portion 402 further has a bottom wall 406, which may be thinner than the thickness of wall 404. In other embodiments bottom wall 406 may be about as thick as sidewall 404.

The attachment portion 400 may be molded of a relatively rigid polymer compound such as one containing polyphenylsulfone (PPSU). Male helical threads 412 are used to attach vessel 108 to any of assembly 102, assembly 110 or lid 112.

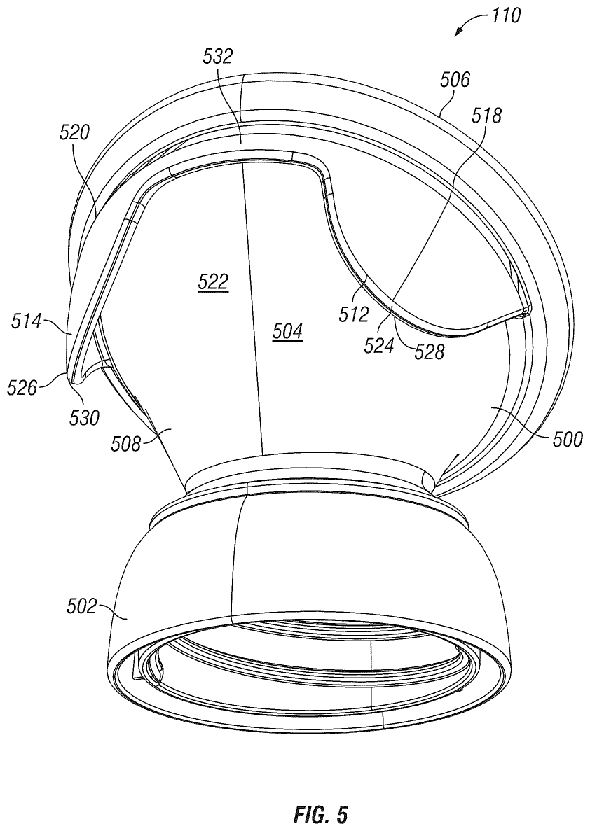

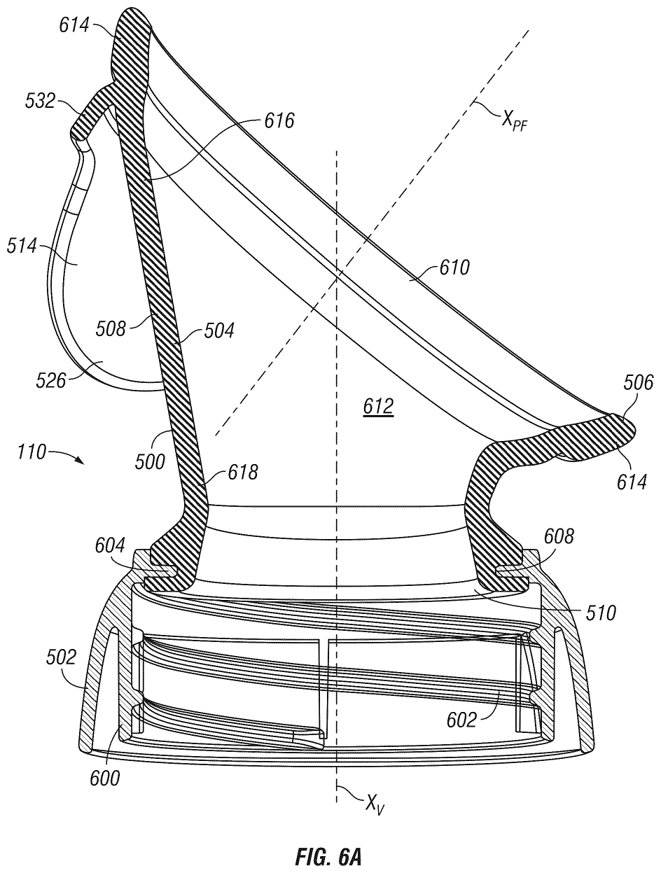

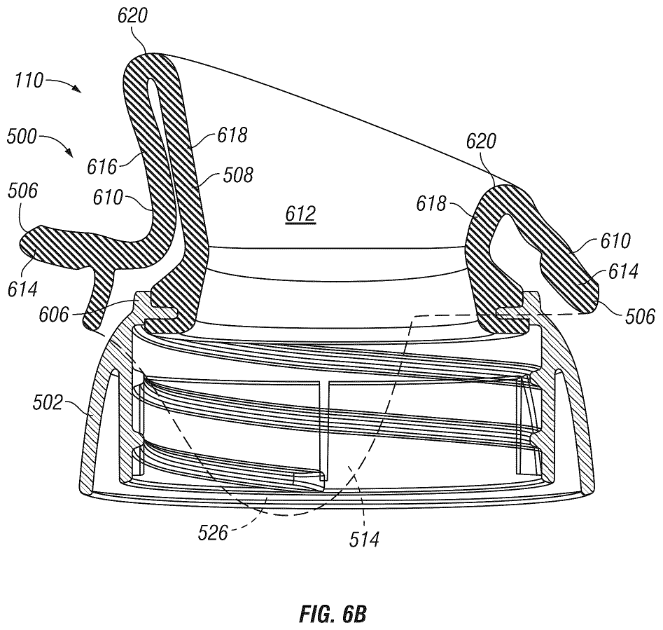

The passive breast pump assembly or passive top 110 is shown in more detail in FIGS. 5, 6A and 6B. Passive top 110 comprises a flexible breast flange 500 formed of an elastomer such as silicone rubber, and a collar 502 molded of a polymer compound that is more rigid than that used for flange 500. The polymer compound making up collar 502 could, for example, include polypropylene.

Breast flange 500 has a body 504 with an endless curved proximal margin 506 that, for example, may be circular or oval. Body 504 has a body wall 508 that extends from proximal margin 506 to a distal end 510 (FIGS. 6A and 6B). At least one, and in the illustrated embodiment two, tabs 512 and 514 extend outwardly and distally from respective proximal ends 518 and 520 on an outer surface 522 of the body wall 508 to respective free ends 524 and 526. In the illustrated embodiment, the proximal or attached tab ends 518 and 520 are spaced distally from flange proximal margin 506. The tabs 512 and 514 are integrally molded with body 504. In the illustrated embodiment, free ends 524 and 526 are convexly curved in a distal direction, and have points 528 and 530 that are farthest from respective attached tab ends 518 and 520. As measured around a breast flange axis X_{PF} (FIG. 6A), points 528 and 530 are widely angularly spaced apart from each other, and may be angularly spaced apart by about 120 degrees. In the illustrated embodiment, a relatively shallow tab wall 532 (FIG. 5) distally and outwardly extends from outer surface 522, and connects tabs 512 and 514 together. Without being limited by theory, it is believed that wall 532 assists in transferring the load from either of the tabs 512, 514, to flange 500. In other embodiments, wall 532 may be omitted.

As seen in FIGS. 6A and 6B, the collar 502 has an inner cylindrical wall 600 with inwardly extending female helical threads 602. These are adapted to engage with male threads 412 of vessel 108 (FIG. 4). A radially inwardly extending, annular flange 604 at top end 606 of the collar 502 fits into an annular groove 608 formed at distal end 510 of the flange 500. Groove 608 and flange 604 may be attached to each other with a suitable adhesive.

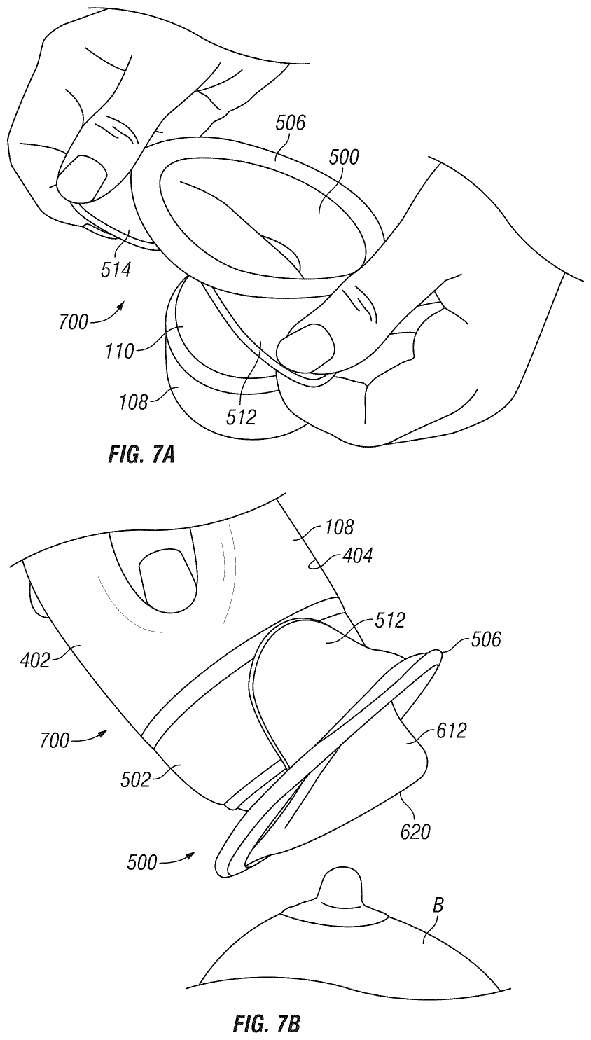

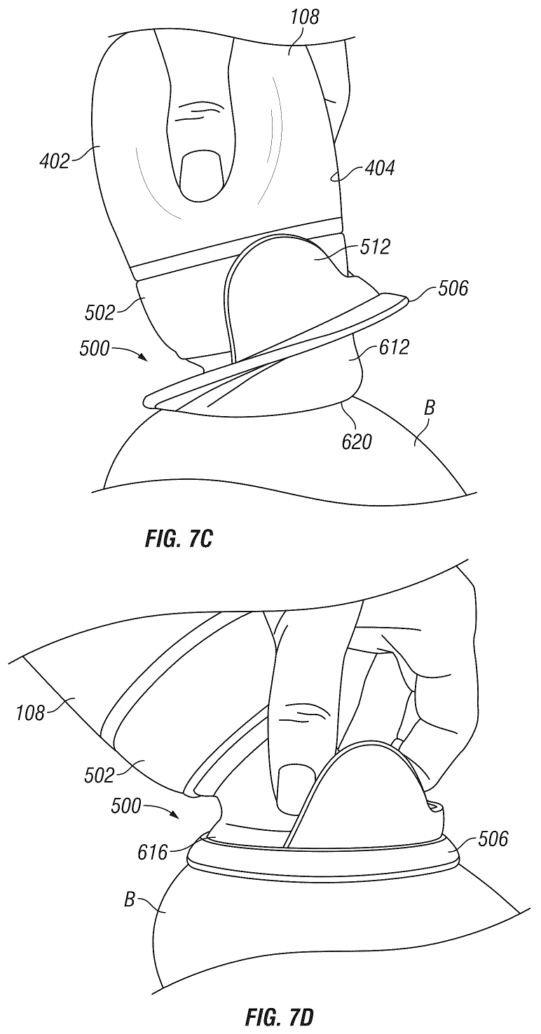

FIGS. 5 and 6A show flange 500 in a stable normal state in which no significant force has been applied to body 504. In this state, the proximal margin 506 may reside in a plane orthogonal to a breast flange axis X_{PF}, which in turn is at an angle to vessel axis X_{V}. In the illustrated embodiment, axes X_{PF} and X_{V} are at about 105 degrees to each other. In the normal state, a proximal portion 610 of a breast flange inner surface 612 will be proximally concavely curved so as to best seal to a woman's breast B (FIG. 7D). The breast flange body 508 may have a somewhat thicker reinforcing ring 614 disposed adjacent proximal margin 506. For example, a thickness of wall 508 may be about 3 mm, while a thickness of reinforcing ring 614 may be about 4 mm to about 5 mm. In the normal state, a proximal portion 616 of the body wall 508 assumes a normal position, in which portion 616 proximally extends from a distal portion 618 thereof to proximal margin 506.

An everted state of breast flange 500 is shown in FIG. 6B. In the illustrated everted state, the proximal portion 616 of wall 508 has been pulled back distally, so as to be disposed radially outwardly from a distal portion 618 of the wall 508. The proximal margin 506 may now be nearer to top end 606 of collar 502 than its disposition in the normal state. The proximal portion 616 assumes an everted position that is distally displaced from its normal position. In the everted state, a locus 620 of the inner surface 612 becomes the new proximal margin of the flange 500 and it is this locus that will be initially pressed to breast B (FIG. 7C). In the illustrated embodiment, the degree to which flange wall 508 may be doubled or peeled back on itself may be somewhat variable, and therefore the location of locus 620 on inner surface 612 may vary likewise. The reinforcing ring 614, because it is thicker, has more hoop strength than the rest of wall 508. This means that it will require more force to temporarily stretch ring 614 in going to and from the everted state, creating a degree of hysteresis and aiding in the stability of both the normal and everted states.

The everted state illustrated in FIGS. 6B, 7B and 7C is stable, in that an external force from a distal direction to wall proximal portion 610 will be necessary to “pop” the flange 500 back to a reinverted or normal state as seen in FIGS. 6A and 7D.

In order to evert the breast flange 500 from a normal state to an everted state, a user applies at least a predetermined tensile force in a distal direction to flange 500 through at least one of tabs 512 and 514, and preferably through both of them. For example, and as seen in FIG. 7A, one of the tabs 512, 514 may be grasped with the thumb and fingers of one hand, and the other of tabs 512, 514 may be gasped with the thumb and fingers of the other hand. Using the tabs 512, 514 means that the user does not have to manipulate proximal margin 506 or put her fingers anywhere on inner flange surface 612 to evert flange 500, enhancing cleanliness and removing a possible source of contamination of the expressed milk. The tabs 512, 514 are also more convenient to manipulate than proximal margin 506, particularly of a margin 506 that has been reinforced with ring 614.

Because the attachment portion 400 of vessel 108 is rigid, it is suitable as a component of a lever-operated manual breast pump that also includes lever-operated breast pump assembly 102. But, because it has a resilient flexible sidewall 404, it is also suitable for being a component of a passive manual breast pump 700, as seen in use in FIGS. 7A-7D, and as including the passive breast pump assembly 110.

Steps in applying a passive manual breast pump to a user's breast are shown in FIGS. 7A-7D. In FIG. 7A, a user has screwed the passive breast pump assembly onto a vessel 108 to form the passive manual breast pump 700. The bottom wall 406 of the vessel 108 may be placed on a stationary surface such as a horizontal surface. The user grasps the tab 512 with the thumb and fingers of one hand, and grasps the tab 514 with the thumb and fingers of the other hand. The user then pulls the proximal portion 610 of flange wall 508 distally (here, downwardly) until the breast flange 500 assumes an everted state.

This everted state is shown in FIG. 7B. Next, the user squeezes sidewall 404 with her hand, in order to reduce the volume of vessel interior 410.

As shown in FIG. 7C, and while continuing to squeeze vessel sidewall 404, locus 620 of everted breast flange 500 is pressed around the aureole of the user's breast B. This seals the volume inside of vessel 108 and the distal portion 618 of breast flange 500 from outside air.

Finally, and as shown in FIG. 7D, the user applies force from a distal direction to breast flange wall proximal portion 616 to reinvert it to the normal state. Proximal portion 616 “pops” onto an annulus of skin around the breast aureole, creating a long-lasting seal of the inner surface 612 to breast B, and ensuring that a negative vacuum will be experienced at the nipple of the breast, inducing milk flow.

In summary, a novel manual breast pump kit has been shown and described that includes both a lever-operated manual breast pump assembly and a passive breast pump assembly. Characteristics of a vessel comprising a component of the kit make it suitable for use with either. An offset vessel tube of the lever-operated assembly makes the assembly easier to operate by a user with small hands. An elastomeric breast flange of the passive breast pump assembly has tabs to aid in its eversion prior to application to a breast.

While illustrated embodiments of the present invention have been described and illustrated in the appended drawings, the present invention is not limited thereto but only by the scope and spirit of the appended claims.

We claim:

1. A breast flange comprising:
a body formed of an elastomer and having an endless curved proximal margin, a body wall extending from the proximal margin to a distal end of the body, means for communicating the distal end to a milk storage vessel;
the body wall having an outer surface, at least one tab extending from the outer surface to a free end;
the body capable of assuming normal and everted states, each state being stable;
the body wall having a proximal portion and a distal portion, the proximal portion, in the normal state of the body, having a normal position in which the proximal portion proximally extends from the distal portion to the proximal margin;
the proximal portion of the body wall, in the everted state, having an everted position which is outwardly and distally displaced from the normal position so as to double back on the distal portion, a predetermined amount of distal tensile force applied by a user through the at least one tab everting the body from the normal state to the everted state.

2. The breast flange of claim 1, wherein the flange is disposed on a flange axis, the at least one tab being a first tab, a second tab formed on the outer surface of the body wall to be angularly displaced from the first tab.

3. The breast flange of claim 1, wherein the means for communicating the distal end to the milk storage vessel is a collar having a top end and a bottom end, the distal end of the breast flange attached to the top end of the collar, the bottom end of the collar removably attached to the milk storage vessel.

4. A passive breast pump including the breast flange of claim 1, further comprising a milk storage vessel, wherein the milk storage vessel has a vessel sidewall formed of a second elastomer.

5. The passive breast pump of claim 4, wherein the first elastomer and the second elastomer are silicone rubber.

6. The breast flange of claim 1, wherein the proximal margin of the breast flange is circular.

7. The breast flange of claim 1, wherein the proximal end of the body is arranged on a breast flange axis, the distal end of the body formed around a vessel axis disposed at an angle to the breast flange axis.

8. A manual breast pump kit comprising:
a lever-operated breast pump assembly including a body, a diaphragm and a handle, the body being formed of a first polymer and having a vessel collar, a diaphragm chamber of the body bounded by the diaphragm, the diaphragm being formed of a first elastomer and movable between a first position at which the diaphragm chamber has a first volume, and a second position at which the diaphragm chamber has a second volume larger than the first volume, the body having a fulcrum, the handle formed of a second polymer and having a free end and a diaphragm end, a pivot of the handle disposed between the free end and the diaphragm end and rotatably coupled to the fulcrum of the body, a diaphragm link coupling the diaphragm end of the handle to the diaphragm and operable to displace the diaphragm from the first position to the second position, the first and second polymers being sufficiently rigid to permit a hand of a user to displace the free end of the handle toward the vessel collar and thereby urge the diaphragm from the first position to the second position;
a vessel selectively attachable to the vessel collar of the lever-operated breast pump assembly, a sidewall of the vessel being formed of a second elastomer that is more flexible than the first or second polymers, the vessel having a vessel interior; and
a passive breast pump assembly comprising a flexible breast flange molded of a third elastomer, a proximal end of the second breast flange adapted to fit to a breast of a user, the second breast flange having a distal end, a second collar of the passive breast pump assembly formed from a third polymer that is more rigid than the second elastomer and the third elastomer, the second collar joined to the flexible breast flange and operable to be fastened to the vessel so as to communicate the distal end of the flexible breast flange to the vessel interior, whereby, when the passive breast pump assembly is fastened to the vessel, the user may squeeze the sidewall of the vessel to create a partial vacuum at the proximal end of the second breast flange.

9. The kit of claim 8, wherein the second elastomer is silicone rubber.

10. The kit of claim 8, wherein the third elastomer is silicone rubber.

11. The kit of claim 8, wherein the first polymer is polypropylene.

12. The kit of claim 8, wherein the second polymer is polypropylene.

13. The kit of claim 8, wherein the third polymer is polypropylene.

14. The kit of claim 8, wherein the vessel comprises an attachment portion for selective attachment to the vessel collar of the lever-operated breast pump assembly or to the second collar of the passive breast pump assembly, and a flexible portion, the flexible portion including said sidewall of the vessel, the attachment portion being formed of a fourth polymer that is more rigid than the second elastomer.

15. The kit of claim 14, wherein the attachment portion of the vessel is molded from polyphenylsulfone.

16. A manual breast pump kit comprising:
a lever-operated breast pump assembly including a body, the body having a breast flange tube;
a plurality of breast flanges each selectable by the user for attaching to the breast flange orifice, each breast flange having a proximal margin for fitting to the user's breast, a flange portion extending distally from the proximal margin to a distal end of the flange portion, and a tube portion distally extending from the distal end of the flange portion to a tube end, the tube portion adapted to be inserted into the breast flange tube of the body, the tube portion connected to the distal end of the flange portion at a transition, a distal end internal diameter taken at the transition;
the distal end internal diameter of a first of the plurality of breast flanges being different from the distal end internal diameter of a second of the plurality of breast flanges, such that differences in nipple sizes are accommodated by different ones of the plurality of breast flanges.

17. The kit of claim 16, wherein the plurality of breast flanges further includes a third breast flange, the distal end internal diameter of the third breast flange being different from the distal end internal diameters of the first and second breast flanges.

18. The kit of claim 16, wherein at least one of the plurality of breast flanges has a proximal margin which is oval.

19. A vessel for containing breast milk comprising:
an attachment portion for attachment to a breast pump assembly, the attachment portion molded of a first polymer; and
a flexible portion overmolded onto the attachment portion, the flexible portion being molded of an elastomer that is less rigid than the first polymer, the flexible portion including a flexible sidewall which will inwardly elastically deform when squeezed by a hand of a user, the flexible sidewall defining a vessel interior, the flexible portion having an original state, such that, when the vessel interior is in communication with the atmosphere, and when the hand of the user is no longer squeezing the flexible sidewall, the flexible sidewall will return to its original state.

20. The vessel of claim 19, wherein the flexible sidewall is formed around an axis and has a top end and a bottom end, the flexible sidewall having an inner surface and a vessel diameter taken at the inner surface of the sidewall across the axis at a point about midway between the top end and the bottom end, the flexible sidewall being capable of being compressed by 95% of adult female users radially inwardly toward the axis by at least 50% of the vessel diameter.

21. The vessel of claim 19, wherein the flexible sidewall is formed around an axis and has a top end and a bottom end, the flexible sidewall having an inner surface and a vessel diameter taken at an inner surface of the sidewall across the axis at a point about midway between the top end and the bottom end, the flexible sidewall being capable of being compressed by 80% of adult female users radially inwardly toward the axis by at least 50% of the vessel diameter.

22. The vessel of claim 19, wherein the elastomer is silicone rubber.

23. The vessel of claim 19, wherein the attachment portion has an external cylindrical surface with helical threads.

24. The vessel of claim 19, wherein the attachment portion is integrally molded from polyphenysulfone.

25. A lever-operated breast pump comprising:
a vessel formed around a vessel axis and having an opening;
a lever-operated breast pump assembly including a body, a breast flange and a pumping lever;
the body including a breast flange tube in fluid communication with the breast flange, the breast flange tube disposed at an angle to the axis, the breast flange tube having a lower outer surface, a vessel tube of the body in fluid communication with the breast flange tube, the vessel tube disposed on a vessel tube axis parallel to but horizontally offset from the vessel axis in a first horizontal direction, the vessel tube having an outer surface facing in a second direction opposite the first direction, said outer surface of the vessel tube being a thumb engagement surface;
a downwardly hollow vessel collar of the body joined to the vessel tube and adapted to be affixed to the opening of the vessel, the collar having an upper surface downwardly spaced from the lower outer surface of the breast flange tube, the lower outer surface of the breast flange tube, the thumb engagement surface of the vessel tube and the upper surface of the collar defining a thumb space capable of receiving a thumb of a user;
the pumping lever having a free end, a diaphragm end and a pivot disposed between the free end and the diaphragm end, a grasping surface of the pumping lever having a first end proximate the pivot and extending generally downwardly from the first end to the free end and adapted to receive one or more fingers of the user, clamping pressure exerted by the fingers of the user on the grasping surface causing displacement of the grasping surface in the second direction toward the thumb space.

26. The breast pump of claim 25, wherein the vessel tube axis is displaced from the vessel axis by a displacement selected from a range of about 3 mm to about 16 mm.

27. The breast pump of claim 26, wherein the displacement is about 7 mm.

28. The breast pump of claim 25, wherein the grasping surface has a substantially vertical segment, the vertical segment having a rest state, a distance between the thumb engagement surface of the vessel tube and the vertical segment in the rest state being selected from a range of about 60 mm to about 100 mm.

29. The breast pump of claim 28, wherein the distance between the thumb engagement surface and the vertical segment of the grasping surface in the rest state is about 70 mm.

30. A method of using a passive manual breast pump, the method comprising the steps of:
providing an elastomeric breast pump flange with at least one tab outwardly and distally extending from a location on an outer surface of a breast flange wall;
applying a predetermined amount of tensile force in a distal direction to the at least one tab;
responsive to the said step of applying, everting a proximal portion of the breast flange wall so that the proximal portion is outwardly and distally displaced from a normal position of the proximal portion, the breast flange thereby assuming an everted state;
squeezing an elastomeric sidewall of a vessel in fluid communication with the breast flange;
while performing said step of squeezing, applying a locus on an internal surface of the breast flange to the breast, the locus being a proximal end of the breast flange when the breast flange is in the everted state; and
after said step of applying, exerting force in a proximal direction on the proximal portion of the breast flange wall, to thereby reinvert the breast flange to a normal state, seal the internal surface of the flange to the breast, and maintain a partial vacuum at the breast.
